(19) United States
(12) Patent Application Publication
(10) Pub. No.: US 2017/0247429 A1
(43) Pub. Date: Aug. 31, 2017

(54) BLOOD-BRAIN BARRIER DISRUPTING AGENTS AND USES THEREOF

(71) Applicants: TEL HASHOMER MEDICAL RESEARCH INFRASTRUCTURE AND SERVICES LTD., Ramat Gan (IL); YEDA RESEARCH AND DEVELOPMENT CO. LTD., Rehovot (IL)

(72) Inventors: Itzik Cooper, Rehovot (IL); David Guez, Tel Aviv (IL); David Last, Jerusalem (IL); Yoram Shechter, Rehovot (IL); Matityahu Fridkin, Rehovot (IL); Yael Mardor, Netanya (IL)

(21) Appl. No.: 15/505,526

(22) PCT Filed: Sep. 16, 2015
(86) PCT No.: PCT/IL2015/050939

§ 371 Date: Feb. 21, 2017

Related U.S. Application Data
(60) Provisional application No. 62/051,975, filed on Sep. 18, 2014.

(51) Int. Cl. C07K 14/765; A61K 9/00; A61K 45/06

(52) U.S. Cl. CPC C07K 14/765; A61K 47/48284; A61K 45/06; A61K 9/0085

(57) ABSTRACT

The present invention relates to blood-brain barrier disrupting agents containing a modified serum albumin comprising serum albumin. The present invention further relates to pharmaceutical compositions comprising said agents and use thereof for the treatment of brain diseases and disorders.

### FIELD OF THE INVENTION

The present invention relates to blood-brain barrier disrupting agents. The present invention further relates to pharmaceutical compositions comprising said agents and use thereof for the treatment of brain diseases and disorders.

### BACKGROUND OF THE INVENTION

Brain tumors belong to one of the most lethal types of cancer. In the United States alone, more than 700,000 people have been diagnosed with primary brain or central nervous system (CNS) tumor. It is reported that only five percent of diagnosed patients will survive beyond five years. Malignant gliomas are the most common type of primary malignant brain tumor, accounting for 80% of patients.

The current therapy for treating brain tumors primarily includes drugs having a low penetration across the blood-brain barrier (BBB). Consequently, the common administration protocols involve administering systemically high doses of chemotherapeutics in an attempt to reach therapeutically effective intracranial therapeutic concentrations. These attempts resulted in systemic toxicity and serious adverse effects. Although the BBB is compromised to some extent in malignant gliomas, the resulting permeability is not sufficient for delivering therapeutic doses of drugs to the tumor tissues via systemic routes. Moreover, the BBB in the infiltrating zone surrounding the tumor mass remains mostly intact, thus, restricting the penetration of drugs into these regions. For treatment to be effective, it is necessary that therapeutic drug doses would access the entire tumor and its vicinity. Survival of even a few cancerous cells may result with cancer reoccurrence, a prevailing phenomenon with high-grade gliomas.

Cationized albumin was found to induce BBB disruption in vitro (Cooper et al., J. Biol. Chem., 2012, 287: 44676-44683). However, studies report that cationized albumin is heavily taken by the kidneys and liver when systemically administered (Bregmann et al., Clin. Sci., 1984, 67: 35-43).

There remains an unmet need to develop novel BBB penetrating agents that can induce a significant yet transient local BBB disruption in brain pathology and surrounding infiltration zone.

### SUMMARY OF THE INVENTION

The present invention provides blood-brain barrier disrupting agents comprising chemically modified serum albumin. The present invention further provides pharmaceutical compositions comprising said agents and use thereof for the treatment of brain diseases and disorders, including brain tumors, such as, glioblastoma multiforme (GBM), meningioma and oligodendrogliomas.

The present invention is based in part on the unexpected discovery that neutralized serum albumin exhibit an efficient, transient and safe local BBB disruption in rat glioma brain tumor models. Furthermore, conjugates comprising neutralized HSA covalently bound to chemotherapy as well as compositions of neutralized HSA in combination with a chemotherapeutic agent, where shown to disrupt the BBB, thereby enabling delivery of the chemotherapeutic agent to the brain. Surprisingly, it was further found that intracranial-convection-enhanced-delivery (CED) is the optimal route of administration of the compounds of the invention, providing maximal BBB disruption and minimal brain toxicity. Another unexpected findings on which the present invention is founded, is that convection-enhanced delivery of the BBB disrupting agents of the invention in combination with systemic administration of anti-cancer therapy, in vivo, suppresses tumor growth resulting with a significantly prolonged survival. Thus, the compounds and administration protocols of the invention provide effective, minimally-invasive and safe therapy, thereby offering a new approach for overcoming the drawbacks and deficiencies of the known treatments.

According to some embodiments the present invention provides a modified serum albumin comprising serum albumin, or an analogue thereof, having a plurality of neutralized amino acid side chain residues selected from Aspartic acid side chain residue, Glutamic acid side chain residue and a combination thereof, wherein each of said neutralized amino acid side chain residues is covalently attached to a capping moiety.

According to some embodiments, the serum albumin is human serum albumin.

According to some embodiments, the capping moiety comprises a linear or branched C1-C8 alkyl, C2-C8 alkenyl, C2-C8 alkynyl, C3-C8 cycloalkyl, heterocyclyl, heteroaryl, aryl, which is substituted with one or more substituents selected from the group consisting of: —X, —O—, —S—, —SH, —NH—, —NH₂, —C(═O)—, —C(═O)X, —C(═O)OC(═O)—, —C(═O)O—, —OC(═O)O—, —C(═O)NH—, —C(═O)NH₂, —NHC(═O)NH—, —NHC(═O)O—, —S(═O)—, —S(═O)O—, —PO(═O)O— or any combination thereof.

According to some embodiments, the capping moiety comprises a nitrogen containing substituent. According to some embodiments, the nitrogen containing substituent is covalently connected to the albumin through said nitrogen. According to some embodiments, the nitrogen containing substituent is covalently connected to the albumin through an amide bond between the nitrogen atom of the nitrogen containing substituent and a carbonyl moiety of amino acid side chain residues of the albumin.

According to some embodiments, the capping moiety is a primary amine. According to some embodiments, the capping moiety is selected from the group consisting of glycine amide, alanine amide, leucine amide, ethylamine, propylamine and ethanol amine.

According to some embodiments, the capping moiety is ethylamine. According to some embodiments, the capping moiety is glycine amide. According to some embodiments, the capping moiety is alanine amide. According to some embodiments, the capping moiety is leucine amide. According to some embodiments, the capping moiety is propylamine. According to some embodiments, the capping moiety is ethanol amine.

According to some embodiments the present invention provides a pharmaceutical composition comprising a modified serum albumin comprising serum albumin, or an analogue thereof, having a plurality of neutralized amino acid side chain residues selected from Aspartic acid side chain residue, Glutamic acid side chain residue and a combination thereof, wherein each of said neutralized amino acid side chain residues is covalently attached to a capping moiety, and further comprising pharmaceutically acceptable diluents or carriers.

According to some embodiments the present invention provides a pharmaceutical composition comprising a cationized serum albumin or an analogue thereof, said cationized serum albumin comprises a plurality of cationized amino acid side chain residues selected from Aspartic acid side chain residue, Glutamic acid side chain residue and a combination thereof, and further comprising pharmaceutically acceptable diluents or carriers.

According to some embodiments, the modified serum albumin further comprises at least one therapeutic agent moiety covalently attached to the albumin through a lysine side chain residue, thereby producing a conjugate. According to some embodiments, the cationized serum albumin further comprises at least one therapeutic agent moiety covalently attached to the albumin through a lysine side chain residue, thereby producing a conjugate.

According to some embodiments, the therapeutic agent is covalently connected to the lysine ε-amino group. According to some embodiments the therapeutic agent comprises a prodrug. According to some embodiments the therapeutic agent is a prodrug. According to some embodiments the therapeutic agent comprises a drug. According to some embodiments the therapeutic agent comprises a chemotherapeutic agent. According to some embodiments the therapeutic agent is released from the albumin through metabolism. According to some embodiments the therapeutic agent is released from the albumin inside the cell. According to some embodiments the conjugate is degraded following internalization into cells, thereby releasing the therapeutic agent from the albumin within the cell. According to some embodiments the drug comprises MTX.

According to some embodiments, the present invention provides a method for increasing BBB permeability in a subject in need thereof comprising administering to the subject a pharmaceutical composition comprising a modified serum albumin comprising serum albumin, or an analogue thereof, having a plurality of neutralized amino acid side chain residues selected from Aspartic acid side chain residue, Glutamic acid side chain residue and a combination thereof, wherein each of said neutralized amino acid side chain residues is covalently attached to a capping moiety.

According to some embodiments the present invention provides a method for increasing BBB permeability in a subject in need thereof comprising administering to a subject the pharmaceutical composition comprising a serum albumin or an analogue thereof, said serum albumin comprises a plurality of cationized amino acid side chain residues selected from Aspartic acid side chain residue, Glutamic acid side chain residue and a combination thereof.

According to some embodiments, the method further comprises administering to said subject at least one therapeutic agent.

According to some embodiments, the present invention provides a method for treating a disease or disorder in a subject in need thereof comprising administering to said subject a pharmaceutical composition comprising a modified serum albumin comprising serum albumin, or an analogue thereof, having a plurality of neutralized amino acid side chain residues selected from Aspartic acid side chain residue, Glutamic acid side chain residue and a combination thereof, wherein each of said neutralized amino acid side chain residues is covalently attached to a capping moiety; and administering to said subject at least one therapeutic agent.

According to some embodiments, the present invention provides a method for treating a disease or disorder in a subject in need thereof comprising administering to said subject a pharmaceutical composition comprising a serum albumin or an analogue thereof, said serum albumin comprises a plurality of cationized amino acid side chain residues selected from Aspartic acid side chain residue, Glutamic acid side chain residue and a combination thereof; and administering to said subject at least one therapeutic agent.

According to some embodiments, the at least one therapeutic agent is selected from the group consisting of anti-neoplastic agents, anti-angiogenic agents, siRNAs, immuno-therapeutic agents and chemotherapeutic agents.

According to some embodiments, the at least one therapeutic agent is an antimetabolite.

According to some embodiments, said at least one therapeutic agent is an anti-neoplastic agent.

According to some embodiments, said pharmaceutical composition is administered intracranially. According to some embodiments, said pharmaceutical composition is administered intracranially by convection-enhanced delivery.

According to some embodiments, said at least one therapeutic agent is administered via an administration route selected from the group consisting of systemic, intraperitoneal, intracranial and intravascular administration. According to some embodiments, said at least one therapeutic agent is administered intracranially by convection-enhanced delivery.

According to some embodiments, said administering at least one therapeutic agent is performed simultaneously or subsequently to said administering the pharmaceutical composition. According to some embodiments, said administering at least one therapeutic agent is performed subsequently to said administering the pharmaceutical composition. According to some embodiments, said administering at least one therapeutic agent is performed simultaneously with said administering the pharmaceutical composition.

According to some embodiments the present invention provides a use of a pharmaceutical composition comprising a modified serum albumin comprising serum albumin, or an analogue thereof, having a plurality of neutralized amino acid side chain residues selected from Aspartic acid side chain residue, Glutamic acid side chain residue and a combination thereof, wherein each of said neutralized amino acid side chain residues is covalently attached to a capping moiety, for increasing BBB permeability.

According to some embodiments the present invention provides a use of a pharmaceutical composition comprising a serum albumin or an analogue thereof, said serum albumin comprises a plurality of cationized amino acid side chain residues selected from Aspartic acid side chain residue, Glutamic acid side chain residue and a combination thereof, for increasing BBB permeability.

According to some embodiments, the use of said pharmaceutical composition is by intracranial convection-enhanced delivery.

According to some embodiments, the use of said pharmaceutical composition is in combination with at least one therapeutic agent.

According to some embodiments the present invention provides a use of a pharmaceutical composition comprising a modified serum albumin comprising serum albumin, or an analogue thereof, having a plurality of neutralized amino acid side chain residues selected from Aspartic acid side chain residue, Glutamic acid side chain residue and a combination thereof, wherein each of said neutralized amino acid side chain residues is covalently attached to a capping moiety, in combination with at least one therapeutic agent, for the treatment of a disease or disorder.

According to some embodiments the present invention provides a use of a pharmaceutical composition comprising a serum albumin or an analogue thereof, said serum albumin comprises a plurality of cationized amino acid side chain residues selected from Aspartic acid side chain residue, Glutamic acid side chain residue and a combination thereof, together with at least one therapeutic agent, for the treatment of a disease or disorder.

According to some embodiments the present invention provides a kit for increasing BBB permeability comprising at least one first container comprising a pharmaceutical composition comprising a modified serum albumin comprising serum albumin, or an analogue thereof, having a plurality of neutralized amino acid side chain residues selected from Aspartic acid side chain residue, Glutamic acid side chain residue and a combination thereof, wherein each of said neutralized amino acid side chain residues is covalently attached to a capping moiety.

According to some embodiments the present invention provides a kit for increasing BBB permeability comprising at least one first container comprising a pharmaceutical composition comprising a serum albumin or an analogue thereof, said serum albumin comprises a plurality of cationized amino acid side chain residues selected from Aspartic acid side chain residue, Glutamic acid side chain residue and a combination thereof.

According to some embodiments the present invention provides a kit for treating brain disease or disorder comprising at least one first container comprising a pharmaceutical composition comprising a modified serum albumin comprising serum albumin, or an analogue thereof, having a plurality of neutralized amino acid side chain residues selected from Aspartic acid side chain residue, Glutamic acid side chain residue and a combination thereof, wherein each of said neutralized amino acid side chain residues is covalently attached to a capping moiety; and at least one second container comprising at least one therapeutic agent.

According to some embodiments the present invention provides a kit for treating brain disease or disorder comprising at least one first container comprising a pharmaceutical composition comprising a serum albumin or an analogue thereof, said serum albumin comprises a plurality of cationized amino acid side chain residues selected from Aspartic acid side chain residue, Glutamic acid side chain residue and a combination thereof; and at least one second container comprising at least one therapeutic agent.

According to some embodiments, treating a disease or disorder comprises increasing BBB permeability.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1B show the barrier disruption in-vitro as a function of time (hr), reflected by the percentage reduction in TEER (trans endothelial electrical resistance) value (TEER at time 0>300 Ωcm²) in the presence/absence of EA-HSA (control diamond; 0.2 mg/ml square; 0.4 mg/ml triangle; 0.8 mg/ml cross; and 1.6 mg/ml asterisk) (A) In-vitro permeability (Pe; cm/sec×10⁶) of methotrexate (1 mM) to the abluminal side in control (non-treated) PBEC-M (left column) and in EA-HSA treated PBEC-M (14 μM, 2 hours at 37° C., right column; ***p<0.001) (B).

FIG. 1C is a schematic description of the blood-brain barrier reflecting in-vitro experimental system.

FIG. 2 shows the percentage of glioma cell (CNS-1) viability at the ‘brain’ side (FIG. 1C) following treatment of PBEC-M with EA-HSA (left column), MTX (middle column) and combine therapy (EA-HSA and MTX; right column) at the ‘blood’ side in the ‘brain-cancer related’ in vitro experimental system (***p<0.001).

FIGS. 3A-3B show expression of tight junction proteins in PBEC-M without treatment (control) and following treatment with four different concentration of EA-HSA for 2 hr. Immunostaining of Zonula occludens-1 (ZO-1) and occludin was performed with rabbit anti ZO-1 and mouse anti-occludin, as well as with Cy3-labeled anti-rabbit or Alexa-Flour 488 anti-mouse as secondary antibodies, respectively. Nuclei were counterstained with Hoechst reagent (A). Zoomed region of the merged picture taken from the 0.4 mg/ml treated group demonstrating the migration of occludin from the cell borders into the cytoplasm (B). Representative pictures are displayed from five different experiments. Bar 20 μm. 250×350 mm (300×300 DPI).

FIG. 4 shows stress fibers formation in PBEC-M without treatment (control) and following treatment with two different concentration of EA-HSA for 2 hr. Immunostaining of ZO-1 in actin filaments was performed with rabbit anti ZO-1 as well as with Cy3-labeled anti-rabbit and Alexa Fluor 488-conjugated phalloidin as secondary antibodies, respectively. Nuclei were counterstained with Hoechst reagent. Representative pictures are displayed from four different experiments. Bar 20 μm. 335×189 mm (300×300 DPI)

FIGS. 5A-5D show MR images following intracranial CED administration of EA-HSA in naïve rats. EA-HSA at 20 μg/rat was infused into the rats brains. Shown are T1-weighted MR images acquired 30 min after treatment (A). Gradient echo MR image acquired immediately post treatment (B). T2-weighted images acquired immediately following treatment (C) and T2-weighted images acquired 7 days following treatment (D). The arrows in each picture indicates BBB-disruption (A), lack of hemorrhages (B) and tissue damage (C) or tissue toxicity (D) following one week.

FIGS. 6A-6B show rates of tumor growth in the glioma rat model at Day 2 (A; *p<0.05) and Day 7 (B; ***p<0.001) without treatment (control; n=7; left column), with MTX treatment (MTX; n=10; middle column) and with combined EA-HSA-MTX therapy (MTX+BBB; n=9; right column). Tumor volumes were calculated from the T1-weighted MR images and normalized to the tumor volumes at Day 0. Number of animals reduced in time is indicated in the FIG. 6B.

FIG. 7 is a Kaplan-Meier graph demonstrating survival of glioma rat untreated (Control; square), treated with MTX (MTX; diamond; p<0.001), or combined EA-HSA-MTX therapy (MTX+BBB; triangle; p<0.001).

FIGS. 8A-8B show the barrier disruption in-vitro as a function of time (hr), reflected by the percentage reduction in TEER value (TEER at time 0>300 Ωcm²) in the presence/absence of 1,3-DAP-cationized-HSA (control diamond; 0.5 mg/ml square; and 1.0 mg/ml triangle) (A) In-vitro permeability (Pe; cm/sec×10⁶) of methotrexate (1 mM) to the abluminal side in control (non-treated) PBEC-M (left column) and in 1,3-DAP-cationized-HSA treated PBEC-M (14 μM, 2 hours at 37° C., right column; ***p<0.001) (B).

FIGS. 9C-9F show MR images following intracranial CED administration of 1,3-DAP-cationized-HSA in näive rats. 1,3-DAP-cationized-HSA at 20 μg/rat was infused into the rats brains. Shown are T1-weighted MR images acquired 30 min after treatment (C). Gradient echo MR image acquired immediately post treatment (D). T2-weighted images acquired immediately following treatment (E) and T2-weighted images acquired 7 days following treatment (F). The arrows in each picture indicates BBB-disruption (C), lack of hemorrhages (D) and tissue damage (E) or tissue toxicity (F) following one week.

FIGS. 10A-10B show T1-weighted MRI scans of naïve rats brain 30 min after intracranial CED administration of HSA-Gly₈₅-MTX₃ (40 μg/rat).

### DETAILED DESCRIPTION OF THE INVENTION

The present invention relates to blood-brain barrier disrupting agents and use of same for intracranial therapy of diseases and disorders, including brain tumors.

According to some embodiments, the present invention provides a modified serum albumin comprising serum albumin or an analogue thereof, said serum albumin comprises a plurality of neutralized amino acid side chain residues selected from Aspartic acid side chain residue, Glutamic acid side chain residue and a combination thereof, wherein each of said neutralized amino acid side chain residues is covalently attached to a capping moiety.

The terms “albumin” and “serum albumin”, as used herein, are interchangeable and refer to a major protein component of blood plasma, of about 68,000-69,000 Da.

The terms “attached”, “linked” and “bound” are interchangeable and refer to a chemical bond between two moieties, for example, between albumin and a therapeutic agent or moiety.

Although the methods and compositions of the present invention all refer to human serum albumin (HSA), it will be understood by those skilled in the art that serum albumin from other sources may also be used, such as bovine.

The serum albumin may be an isolated protein or a synthetic protein.

The modified serum albumin of the present invention may be prepared as described in the Example section hereinbelow.

The term “about” as used herein means approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, optionally, 10 percent, up or down (higher or lower).

The term “plurality” as used herein refers to two or more.

The term “neutralized” and “neutralization” as used herein, refers to a chemical modification which renders a previously acidic chemical moiety, in particular an organic carboxylic acid moiety, non-acidic. For example, the esterification or amidation of an organic carboxylic acid constitutes neutralization of a molecule, as the newly formed COOR or CONR′R″ (wherein R is a carbon-linked substituent, and wherein R′ and R″ are hydrogen or carbon-linked substituent) are less acidic than the parent carboxylic acid. Neutralized molecules, specifically proteins having a plurality of side chain residues comprising carboxylic acids, may include complete neutralization of all side chain residues comprising carboxylic acids or partial neutralization of some of the side chain residues.

The term “capping moiety” as used herein, refers to non-immunogenic small chemical moiety. According to some embodiments, the capping moiety is non-immunogenic even when covalently attached to the protein. According to some embodiments, the capping moiety is not a therapeutic agent. According to some embodiments, the capping moiety is not labeled and/or does not include a moiety used for detection purposes, such as, by magnetic resonance- or X-ray-based imaging, for example, MRI or CT.

The capping moiety comprises a linear or branched C1-C8 alkyl, C2-C8 alkenyl, C2-C8 alkynyl, C3-C8 cycloalkyl, heterocyclyl, heteroaryl, aryl, which is substituted with one or more substituents selected from the group consisting of —X (halogen), —O—, —OH, —S—, —SH, —NH—, —NH₂, —C(═O)—, —C(O)H, —C(═O)X, —C(═O)OC(═O)—, —C(═O)O—, —OC(═O)O—, —C(═O)NH—, —C(═O)NH₂, —NHC(═O)NH—, —NHC(═O)O—, —S(═O)—, —S(═O)O—, —PO(═O)O—, —NO₂, —CN, and any combination thereof. Each possibility is a separate embodiment of the invention.

The term “C1-C8 alkyl” as used herein refers to any saturated aliphatic hydrocarbon of 1 to 10 carbon atoms. Examples of alkyl groups include but are not limited to methyl, ethyl, propyl, isopropyl, n-butyl, sec-butyl, t-butyl and the like.

The term “C2-C8 alkenyl” as used herein refers to an aliphatic hydrocarbon group containing at least one carbon-carbon double bond including straight-chain and branched-chain groups. Exemplary alkenyl groups include ethenyl, propenyl, n-butenyl, i-butenyl, 3-methylbut-2-enyl, n-pentenyl and the like.

The term “C2-C8 alkynyl” as used herein refers to an aliphatic hydrocarbon group containing at least one carbon-carbon triple bond including straight-chain and branched-chain groups. Exemplary alkynyl groups include ethynyl, propynyl, n-butynyl, 2-butynyl, 3-methylbutynyl, n-pentynyl, and the like.

The terms “C3-C8 cycloalkyl” used herein generally refer to a C3 to C8 cycloalkyl which includes monocyclic or polycyclic groups. Non-limiting examples of cycloalkyl groups are cyclopropyl, cyclobutyl, cyclopentyl, cyclohexyl or cycloheptyl. The cycloalkyl group can be unsubstituted or substituted with any one or more of the substituents defined above for alkyl.

The term “heterocyclyl” used herein alone or as part of another group denote a five-membered to eight-membered rings that have 1 to 4 heteroatoms, such as oxygen, sulfur and/or nitrogen, in particular nitrogen, either alone or in conjunction with sulfur or oxygen ring atoms. These five-membered to eight-membered rings can be saturated, fully unsaturated or partially unsaturated. Preferred heterocyclic rings include piperidinyl, pyrrolidinyl, pyrrolinyl, pyrazolinyl, pyrazolidinyl, piperidinyl, morpholinyl, thiomorpholinyl, pyranyl, thiopyranyl, piperazinyl, indolinyl, dihydrofuranyl, tetrahydrofuranyl, dihydrothiophenyl, tetrahydrothiophenyl, dihydropyranyl, tetrahydropyranyl and the like.

The term “heteroaryl” used herein alone or as part of another group denotes a heteroaromatic system containing at least one heteroatom ring atom selected from nitrogen, sulfur and oxygen. The heteroaryl generally contains 5 or more ring atoms. The heteroaryl group can be monocyclic, bicyclic, tricyclic and the like. Also included in this expression are the benzoheterocyclic rings. If nitrogen is a ring atom, the present invention also contemplates the N-oxides of the nitrogen containing heteroaryls. Non-limiting examples of heteroaryls include thienyl, benzothienyl, 1-naphthothienyl, thianthrenyl, furyl, benzofuryl, pyrrolyl, imidazolyl, pyrazolyl, pyridyl, pyrazinyl, pyrimidinyl, pyridazinyl, indolyl, isoindolyl, indazolyl, purinyl, isoquinolyl, quinolyl, naphthyridinyl, quinoxalinyl, quinazolinyl, cinnolinyl, pteridinyl, carbolinyl, thiazolyl, oxazolyl, isothiazolyl, isoxazolyl and the like. The heteroaryl group can optionally be substituted through available atoms with one or more groups defined hereinabove for alkyl.

The term “aryl” used herein alone or as part of another group denotes an aromatic ring system containing from 6-14 ring carbon atoms. The aryl ring can be a monocyclic, bicyclic, tricyclic and the like. Non-limiting examples of aryl groups are phenyl, naphthyl including 1-naphthyl and 2-naphthyl, and the like.

“X” as used herein, designates a halogen atom includes chloro, fluoro, bromo, and iodo.

According to some embodiments, the capping moiety comprises a nitrogen containing substituent. According to some embodiments, the nitrogen containing substituent is covalently connected to the albumin through said nitrogen. According to some embodiments, the nitrogen containing substituent is covalently connected to the albumin through an amide bond between the nitrogen atom of the nitrogen containing substituent and a carbonyl moiety of amino acid side chain residues of the albumin.

According to some embodiments, the capping moiety is a primary amine thus forming a secondary amide after its capping to the side chain residues.

The terms “primary amine” and “secondary amide” as used herein, refers to chemicals compounds according to the formulas: RNH₂ and R′CONHR″ respectively, wherein R, R′ and R″ are normally carbon-linked substituents.

According to some embodiments, the capping moiety is selected from a group consisting of glycine amide, β-alanine amide, leucine amide, methylamine, ethylamine, propylamine, butylamine, ethanol amine, ammonia, 2-aminoethylmethyl sulfone, 3-aminopropionaldehyde, N-methylglycinamide, 1-aminopropan-2-one, 2-aminopropanol, 3-methoxypropylamine, monoisopropanolamine and the like. Each possibility is a separate embodiment of the invention.

According to some embodiments, the capping moiety is selected from the group consisting of glycine amide, alanine amide, leucine amide, ethylamine, propylamine and ethanol amine. According to some embodiments, the capping moiety is ethylamine. According to some embodiments, the capping moiety is glycine amide. According to some embodiments, the capping moiety is alanine amide. According to some embodiments, the capping moiety is leucine amide. According to some embodiments, the capping moiety is propylamine. According to some embodiments, the capping moiety is ethanol amine.

The terms “amino acid” and “amino acid residue” are interchangeably and refer to compounds, which have an amino group and a carboxylic acid group, preferably in a 1,2-1,3-, or 1,4-substitution pattern on a carbon backbone. α-Amino acids are most preferred, and include the 20 natural amino acids (which are L-amino acids except for glycine) which are found in proteins, the corresponding D-amino acids, the corresponding N-alkyl amino acids, side chain modified amino acids, the biosynthetically available amino acids which are not found in proteins (e.g., 4-hydroxy-proline(Hyp), 5-hydroxy-lysine (Hyl), 2, 4-diaminobutyric acid (Dab), citrulline(Cit), ornithine (Orn), canavanine, djenkolic acid, β-cyanolanine, and synthetically derived α-amino acids, such as 2-aminoisobutyric acid (AIB), norleucine (Nle), norvaline, homocysteine and homoserine (Hse). β-Alanine and γ-amino butyric acid are examples of 1,3 and 1,4-amino acids, respectively, and many others are well known to the art.

The terms “aspartic acid side chain residue”, “glutamic acid side chain residue” and “lysine side chain residue” as used herein, refers to chemicals moieties according to the formulas: —CH₂COOH; —CH₂CH₂COOH and —CH₂CH₂CH₂CH₂NH₂ respectively. It will be understood by those skilled in the art that the protonated or deprotonated ions corresponding to said moieties (—CH₂COO⁻; —CH₂CH₂COO⁻ and —CH₂(CH₂)₂CH₂NH₃⁺) are also included under the scope of the current invention. It will also be understood by those skilled in the art that the terms “aspartic acid”, “aspartic acid side chain” “aspartate”, “glutamic acid”, “glutamic acid side chain”, “glutamate”, “lysine” and “lysine side chain” may be interchangeable with aspartic acid side chain residue”, “glutamic acid side chain residue” and “lysine side chain residue”.

According to some embodiments, the plurality of neutralized amino acid side chain residues comprises between 1-100, between 10-100, between 20-95, between 30-95, between 35-90, between 40-90 or between 45-85 neutralized amino acid side chain residues. According to some embodiments, the plurality of neutralized amino acid side chain residues comprises at least 50 neutralized amino acid side chain residues. According to some embodiments, the plurality of neutralized amino acid side chain residues comprises at least 60 neutralized amino acid side chain residues. According to some embodiments, the plurality of neutralized amino acid side chain residues comprises at least 70 neutralized amino acid side chain residues.

According to some embodiments, the serum albumin comprises a modified serum albumin comprising serum albumin, or an analogue thereof, having about 70 to about 90 neutralized amino acid side chain residues selected from Aspartic acid side chain residue, Glutamic acid side chain residue and a combination thereof.

The term “cationized albumin” as used herein, refers to serum albumin comprises a plurality of cationized Aspartic acid and/or Glutamic acid side chain residues. Cationized albumin was previously described, for example, Cooper et al. (ibid). In the cationized albumin a plurality of the negatively charged carboxylate moieties of these amino acids is turned into positively charged residue. Typically, this may be achieved by a chemical reaction between the carboxylates and any molecule bearing a functional moiety capable of adhering to the carboxylate which also bears a positive charge, preferably multiply positively charged molecules. Non-limiting examples of such compounds include, 1,3 diaminopropane dihydrochloride, hexamethylenediamine dihydrochloride, cystamine-dihydrochloride, argininamide dihydrochloride and the like. Each possibility is a separate embodiment of the invention.

All stereoisomers, optical and geometrical isomers of the compounds of the instant invention are contemplated, either in admixture or in pure or substantially pure form. The compounds of the present invention can have asymmetric centers at any of the atoms. Consequently, the compounds can exist in enantiomeric or diastereomeric forms or in mixtures thereof. The present invention contemplates the use of any racemates (i.e., mixtures containing equal amounts of each enantiomers), enantiomerically enriched mixtures (i.e., mixtures enriched for one enantiomer), pure enantiomers or diastereomers, or any mixtures thereof. The asymmetric centers can be designated as R/S or as D/L. In addition, several of the compounds of the invention contain one or more double bonds. The present invention intends to encompass all structural and geometrical isomers including cis, trans, E and Z isomers, independently at each occurrence.

One or more of the compounds of the invention, may be present as a salt. The term “salt” encompasses both basic and acid addition salts, including but not limited to phosphate, dihydrogen phosphate, hydrogen phosphate and phosphonate salts, and include salts formed with organic and inorganic anions and cations. Furthermore, the term includes salts that form by standard acid-base reactions of basic groups and organic or inorganic acids. Such acids include hydrochloric, hydrofluoric, hydrobromic, trifluoroacetic, sulfuric, phosphoric, acetic, succinic, citric, lactic, maleic, fumaric, cholic, pamoic, mucic, D-camphoric, phthalic, tartaric, salicyclic, methanesulfonic, benzenesulfonic, p-toluenesulfonic, sorbic, picric, benzoic, cinnamic, and like acids. Additional salts of the compounds described herein may be prepared by reacting the parent molecule with a suitable base, e.g., NaOH or KOH to yield the corresponding alkali metal salts, e.g., the sodium or potassium salts. Additional basic addition salts include ammonium salts (NH₄⁺), substituted ammonium salts, Li, Ca, Mg, salts, and the like.

The present invention also includes solvates of the compounds of the present invention and salts thereof. “Solvate” means a physical association of a compound of the invention with one or more solvent molecules. This physical association involves varying degrees of ionic and covalent bonding, including hydrogen bonding. In certain instances the solvate will be capable of isolation. “Solvate” encompasses both solution-phase and isolatable solvates. Non-limiting examples of suitable solvates include ethanolates, methanolates and the like. “Hydrate” is a solvate wherein the solvent molecule is a water molecule.

The present invention also includes polymorphs of the compounds of the present invention and salts thereof. The term “polymorph” refers to a particular crystalline state of a substance, which can be characterized by particular physical properties such as X-ray diffraction, IR spectra, melting point, and the like.

“Analogs” of serum albumin as used herein, refer to molecules which have the amino acid sequence of serum albumin except for one or more amino acid modifications, including, but not limited to, conservative substitutions of amino acid residues, and optionally one or more peptidomimetic alterations. Analogs are included in the invention as long as they remain pharmaceutically acceptable, and do not confer toxic properties on compositions containing same. The design of appropriate “analogs” may be computer assisted.

The term “Peptidomimetic”, as used herein, refers to serum albumin which is modified in such a way that it includes at least one non-coded residue or non-peptidic bond. Such modifications include, e.g., alkylation and more specific methylation of one or more residues, insertion of or replacement of natural amino acid by non-natural amino acids, replacement of an amide bond with another covalent bond. A peptidomimetic according to the present invention may optionally comprise at least one bond which is an amide-replacement bond such as urea bond, carbamate bond, sulfonamide bond, hydrazine bond, or any other covalent bond.

Conservative substitutions of amino acid residues as known to those skilled in the art are within the scope of the present invention. Conservative amino acid substitutions includes replacement of one amino acid residue with another having the same type of functional group or side chain e.g. aliphatic, aromatic, positively charged, negatively charged. One of skill will recognize that individual substitutions, deletions or additions to protein sequence which alters, adds or deletes a single amino acid or a small percentage of amino acids in the encoded sequence is a “conservatively modified variant” where the alteration results in the substitution of an amino acid residue with a chemically similar amino acid. Conservative substitution tables providing functionally similar amino acids are well known in the art.

According to some embodiments, the modified serum albumin further comprises at least one therapeutic agent moiety covalently attached to the albumin through a lysine side chain residue, thus providing a modified serum albumin-therapeutic agent conjugate.

The terms “therapeutic agent”, “therapeutic moiety” and “therapeutic agent moiety” as used herein are interchangeable and refer to a compound having a therapeutic activity. The compound may include a non-active moiety, such as, a linker, a spacer and the like.

According to some embodiments, said modified serum albumin comprises a plurality of therapeutic agent moieties covalently attached thereto through lysine side chain residues, thus providing a modified serum albumin-therapeutic agent conjugate.

According to some embodiments, said modified serum albumin comprises at least three therapeutic agent moieties covalently attached thereto through lysine side chain residues, thus providing a modified serum albumin-therapeutic agent conjugate.

It will be understood by those skilled in the art that albumin comprises a plurality of amino acid side chain residues, including lysine side chain residue(s), which is linked to a therapeutic agent according to methods known in the art. For example, the linking may include coupling of the lysine side chain residue of the albumin to a carboxylic acid derivative via coupling procedures similar to those known in the art.

According to some embodiments, the present invention provides a conjugate comprising the neutralized or cationized serum albumin of the invention and at least one therapeutic agent moiety covalently attached to the serum albumin through lysine side chain residues.

The term “conjugate” as used herein refers to a compound formed by the joining of two or more chemical compounds. The conjugate may be obtained through the formation of at least one covalent bond between an atom of the first compound and an atom of a second compound. The conjugate may include a spacer/linker moiety, which initially is covalently attached to the first compound and/or the second compound. The linker/spacer may link a plurality of first compounds, such that the conjugate is formed from a single second compound and a plurality of second compounds. Alternatively, the plurality of second compounds may be further linked to one another through a linker/spacer. The conjugate may include, for example, a peptide or a protein and at least one therapeutic agent, such as, a drug molecule.

It will be understood by those skilled in the art that in the preparation of modified serum albumin-therapeutic agent conjugate, each of the modification steps may precede the other. For example, neutralization of the Aspartic acid and/or Glutamic acid side chain residues may be done either prior to or after linking of the therapeutic agent moiety to the albumin's lysine side chain residue(s).

According to some embodiments, the present invention provides a pharmaceutical composition comprising a modified serum albumin comprising serum albumin or an analogue thereof, said serum albumin comprises a plurality of neutralized amino acid side chain residues selected from Aspartic acid side chain residue, Glutamic acid side chain residue and a combination thereof, said pharmaceutical composition further comprises pharmaceutically acceptable diluents or carriers, wherein each of said neutralized amino acid side chain residues is covalently attached to a capping moiety.

According to some embodiments, the present invention provides a pharmaceutical composition comprising a conjugate comprising a modified serum albumin; at least one therapeutic agent moiety covalently attached to the albumin through a lysine side chain residue; and pharmaceutically acceptable diluents or carriers, wherein the modified serum albumin comprises serum albumin or an analogue thereof, said serum albumin comprises a plurality of neutralized amino acid side chain residues selected from Aspartic acid side chain residue, Glutamic acid side chain residue and a combination thereof, such that each of said neutralized amino acid side chain residues is covalently attached to a capping moiety.

According to some embodiments, said conjugate is configured for releasing said at least one therapeutic agent, thus enabling a controlled release of said at least one therapeutic agent in a designated physiological location of the body. According to some embodiments, said designated physiological location is inside a cell. According to some embodiments, said cell is a brain cell. According to some embodiments said conjugate comprises a linker/spacer, which is designed to release said at least one therapeutic agent intracellularly. According to some embodiments, said release is affordable by certain environmental conditions, such as, pH, conductivity, saturation, enzymatic activity and the like.

According to some embodiments, the present invention provides a pharmaceutical composition comprising a modified serum albumin comprising serum albumin or an analogue thereof, said serum albumin comprises a plurality of cationized Aspartic acid and/or Glutamic acid side chain residues and further comprising pharmaceutically acceptable diluents and/or carriers.

The pharmaceutical compositions of the invention may be prepared in any manner well known in the pharmaceutical art.

According to some embodiments, the pharmaceutical composition is in a liquid form such as solution, emulsion or suspension. Each possibility represents as separate embodiment of the present invention.

The term “pharmaceutically acceptable” as used herein means approved by a regulatory agency of the Federal or a state government or listed in the U. S. Pharmacopeia or other generally recognized pharmacopeia for use in animals and, more particularly, in humans.

Useful pharmaceutically acceptable carriers are well known in the art, and include, for example, lactose, glucose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water and methylcellulose. Other pharmaceutical carriers can be sterile liquids, such as water, alcohols (e.g., ethanol) and lipid carriers such as oils (including those of petroleum, animal, vegetable or synthetic origin, such as peanut oil, soybean oil, mineral oil, sesame oil and the like), phospholipids (e.g. lecithin), polyethylene glycols, glycerine, propylene glycol or other synthetic solvents. Each possibility represents as separate embodiment of the present invention.

Pharmaceutical acceptable diluents include, but are not limited to, sterile water, phosphate saline, buffered saline, aqueous dextrose and glycerol solutions, and the like. Each possibility is a separate embodiment of the invention.

According to some embodiments, the present invention provides a method for increasing BBB permeability in a subject in need thereof comprising administering to said subject an effective amount of a pharmaceutical composition comprising modified serum albumin comprising serum albumin or an analogue thereof, said serum albumin comprises a plurality of neutralized amino acid side chain residues selected from Aspartic acid side chain residue, Glutamic acid side chain residue and a combination thereof, said pharmaceutical composition further comprises pharmaceutically acceptable diluents or carriers, wherein each of said neutralized amino acid side chain residues is covalently attached to a capping moiety.

According to some embodiments, the present invention provides a method for increasing BBB permeability in a subject in need thereof comprising administering to said subject an effective amount of a pharmaceutical composition comprising modified serum albumin comprising serum albumin or an analogue thereof, said serum albumin comprises a plurality of cationized amino acid side chain residues selected from Aspartic acid side chain residue, Glutamic acid side chain residue and a combination thereof, said pharmaceutical composition further comprises pharmaceutically acceptable diluents or carriers.

According to some embodiments, the pharmaceutical compositions of the invention are for use in increasing BBB permeability.

The term “increasing BBB permeability” as used herein, refers a significant local BBB disruption in the vicinity of the tumor and infiltrating zone by at least 10%, at least 20%, at least 30%, at least 40%, at least 50%, at least 60% at least 70%, at least 75%, at least 80%, at least 85%, at least 90%, at least 95% or 100%. Each possibility represents as separate embodiment of the present invention.

As demonstrated in the Example section hereinbelow, an efficient and rapid BBB permeability is achieved when about 70% to about 85% of the carboxylate moieties of Aspartic acid and/or Glutamic acid in said serum albumin are neutralized.

According to some embodiments, the method for increasing BBB permeability further comprises administering to said subject at least one therapeutic agent.

According to preferred embodiments, the therapeutic agent comprises methotrexate.

Methotrexate (MTX) is an anti-metabolite agent, a chemical analogue of folic acid. It is used in the present invention as a specific, non-limiting, example of a therapeutic agent. MTX is one of the most widely used drugs for the treatment of many forms of cancer, including tumors of the brain, breast, ovaries, and several leukemias. MTX inhibits folate receptors which are over expressed on the cell membranes of many types of cancer cells.

According to some embodiments, the at least one therapeutic agent is selected from the group consisting of anti-neoplastic agent, anti-angiogenic agent, siRNA, immuno-therapy related agent, growth-inhibitory agent, apoptotic agent, cytotoxic agent and chemotherapeutic agent. Each possibility is a separate embodiment of the invention.

According to some embodiments, the at least one therapeutic agent is a chemotherapeutic agent. According to some embodiments, the at least one therapeutic agent is an antimetabolite.

Examples of therapeutic agents include, but are not limited to, alkylating agents, such as, mustard gas derivatives (Mechlorethamine, cyclophosphamide, chlorambucil, melphalan and ifosfamide), ethylenimines (e.g. Thiotepa and Hexamethylmelamine), alkylsulfonates (Busulfan), hydrazines and triazines (Altretamine, Procarbazine, Dacarbazine and Temozolomide), nitrosoureas (Carmustine, Lomustine and Streptozotocin), ifosfamide and metal salts (Carboplatin, Cis-platin and Oxaliplatin); plant alkaloids, such as, podophyllotoxins (Etoposide and Teniposide), taxanes (Paclitaxel and Docetaxel), vinca alkaloids (Vincristine, Vinblastine, Vindesine and Vinorelbine), and camptothecin analogs (Irinotecan and Topotecan); anti-tumor antibiotics, such as, Chromomycins (Dactinomycin and Plicamycin), anthracyclines (Doxorubicin, Daunorubicin, Epirubicin, Mitoxantrone, Valrubicin and Idarubicin), and miscellaneous antibiotics, such as, Mitomycin, Actinomycin and Bleomycin; anti-metabolites, such as, folic acid antagonists (Methotrexate, Pemetrexed, Raltitrexed, Aminopterin), pyrimidine antagonists (5-Fluorouracil, Floxuridine, Cytarabine, Capecitabine, and Gemcitabine), purine antagonists (6-Mercaptopurine and 6-Thioguanine) and adenosine deaminase inhibitors (Cladribine, Fludarabine, Mercaptopurine, Clofarabine, Thioguanine, Nelarabine and Pentostatin); topoisomerase inhibitors such as topoisomerase I inhibitors (Irinotecan, and Topotecan) and topoisomerase II inhibitors (Amsacrine, Etoposide, Etoposide phosphate, Teniposide); monoclonal antibodies (Alemtuzumab, Gemtuzumab Ozogamicin, Rituximab, Trastuzumab, Ibritumomab tiuxetan, Cetuximab, Panitumumab, Tositumomab, Bevacizumab); and miscellaneous anti-neoplastics, such as, ribonucleotide reductase inhibitors (Hydroxyurea); adrenocortical steroid inhibitor (Mitotane); enzymes (Asparaginase and Pegaspargase); anti-microtubule agents (Estramustine); retinoids (Bexarotene, Isotretinoin, Tretinoin (ATRA) and derivative thereof, and Methotrexate (MTX) among others. Each possibility represents as separate embodiment of the present invention.

According to preferred embodiments, the therapeutic agent is selected from the group consisting of methotrexate, doxorubicin, temozolomide, procarbazine, cis-platin, paclitaxel, docetaxel, and derivative thereof. Each possibility represents as separate embodiment of the present invention.

Non-limiting examples of anti-neoplastic agents that are useful in the present invention including, alkylating agents (e.g. Busulfan, Carbo-platin, Carmustine, Cis-platin, Cyclophosphamide, Dacarbazine, Ifosfamide, Lomustine, Mechlorethamine, Melphalan, Oxaliplatin, Procarbazine, Temozolomide, and Thiotepa); topoisomerase inhibitors (e.g. Dactinomycin, Daunomycin, Doxorubicin, Etoposide, Etoposide phosphate, Idarubicin, Irinotecan, liposomal Daunomycin, liposomal Doxorubicin, Mitoxantrone, Teniposide, and Topotecan); anti-metabolites (e.g. Cytarabine, Clofarabine, Fludarabine, Gemcitabine, Mercaptopurine, Methotrexate, Nelarabine, and Thioguanine); tubulin binders (e.g. Docetaxel, Ixabepilone, Vinblastine, Vincristine, Vinorelbine, and Paclitaxel); molecularly targeted (e.g. Erlotinib, Imatinib, Sorafenib, Sunitinib, Tretinoin, and Herceptin); miscellaneous (e.g. Arsenic trioxide, Asparaginase, Bleomycin, Dexamethasone, Hydroxyurea, Mitotane, PEG-asparaginase, and Prednisone) and derivatives thereof among others. Each possibility is a separate embodiment of the invention.

According to some embodiments, the present invention provides a method for treating a disease or disorder in a subject in need thereof comprising administering to said subject the pharmaceutical compositions of the present invention and, optionally, an additional therapeutic agent.

According to some embodiments, the pharmaceutical compositions of the invention are for treating a brain disease or disorder. According to some embodiments, the pharmaceutical compositions of the invention are for the treatment of brain tumors.

According to some embodiments, the at least one therapeutic agent is administered simultaneously or subsequently to said administering the pharmaceutical composition(s) of the invention. According to some embodiments, the at least one therapeutic agent is administered subsequently to said administering the pharmaceutical composition(s) of the invention. According to some embodiments, the at least one therapeutic agent is administered within 60 minutes, 30 minutes, 15 minutes, 10 minutes or 5 minutes after said administering the pharmaceutical composition(s) of the invention.

It is to be understood that the at least one therapeutic agent may be administered so long that the BBB is open. This may be right after BBB opening is induced by the pharmaceutical composition of the invention, or several minutes thereafter e.g. after 2 minutes, 5, minutes, 10 minutes, 15 minutes, 20 minutes, 25 minutes, 30 minutes, 35 minutes, 40 minutes, 45 minutes, 50 minutes, 55 minutes, 60 minutes or any time during which the BBB is open.

According to some embodiments, the therapeutic agent is administered in a route selected from the group consisting of intracranial, oral, buccal, rectal, transdermal, parenteral (subcutaneous, intraperitoneal, intravenous, intra-arterial, transdermal and intramuscular), topical, or intranasal among others. Each possibility is a separate embodiment of the invention.

According to some embodiments, the pharmaceutical compositions of the invention are administered intracranially. According to some embodiment, the pharmaceutical compositions of the invention are administered intracranially via convection-enhanced delivery.

Without wishing to be limited by any particular theory or mechanism of action, an intracranial administration is especially beneficial for improving efficacy of the composition of the present invention.

Convection-enhanced drug delivery (CED) is a platform for direct delivery of therapeutic agents into the brain. CED includes a continuous infusion of substances via intracranial catheters, leading to convective distribution within the tissue. This approach was found to yield efficient drug distributions at therapeutically effective concentrations in brain tumors, orders of magnitude higher effectivity compared to systemic administration.

According to some embodiments, the method further comprises administering radiation therapy.

As used herein, the term “radiation therapy” including but is not limited to, conventional external radiation therapy, three-dimensional conformal radiation therapy, intensity modulated radiation therapy, stereotactic radiosurgery, fractionated stereotactic radiation therapy, proton radiation therapy, internal, tumor treating fields therapy, and implant radiation therapy among others. Each possibility is a separate embodiment of the invention.

According to some embodiments, each of the pharmaceutical compositions of the invention is administered in a therapeutically effective amount. Typically, the therapeutically effective amounts used according to the teaching of the present invention are lower than the corresponding therapeutically effective amounts required by other methods known in the art, such as, by methods using systemic or intracranial administration of therapeutic agents, devoid of the step of intracranial administration of modified serum albumin.

The term “therapeutically effective amount” as used herein refers to that amount of the pharmaceutical composition being administered which will relieve to some extent one or more of the symptoms of the disease or disorder (e.g. brain tumor) being treated. In reference to cancer or pathologies related to increased cell division, a therapeutically effective amount refers to that amount which has the effect of (1) reducing the size of a tumor, (2) inhibiting (that is, slowing to some extent, preferably stopping) aberrant cell division, (3) preventing or reducing the metastasis of cancer cells, (4) relieving to some extent (or, preferably, eliminating) one or more symptoms associated with a pathology related to or caused in part by unregulated or aberrant cellular division. Each possibility is a separate embodiment of the invention.

The amounts of a modified serum albumin of the invention that are effective in disrupting the BBB and/or treating a disease or disorder, depend on the nature of the disease or disorder, and may be determined by standard non-clinical or clinical techniques. In addition, in vitro assays may optionally be employed to help identify optimal dosage ranges. The precise dose to be employed in the formulation also depends on the route(s) of administration, and the seriousness of the disease, and should be decided according to the judgment of the practitioner and each patient's circumstances. Effective doses may be extrapolated from dose-response curves derived from in vitro or animal model test bioassays or systems.

According to some embodiments, the subject in need thereof is a mammal According to some embodiments, the subject in need thereof is human.

The term “brain tumor” as used herein refers to any one or more of the following: astrocytoma, craniopharyngioma, glioma, ependymoma, neuroglioma, oligodendroglioma, neuroblastoma, glioblastoma (including glioblastoma, multiforme), meningioma, medulloblastoma and other primitive neuroectodermal tumors. Each possibility is a separate embodiment of the invention.

Glioblastoma is the most common and most aggressive malignant primary brain tumor in humans, involving glial cells and accounting for 52% of all functional tissue brain tumor cases and 20% of all intracranial tumors. Without wishing to be limited by any particular theory or mechanism of action, as gliomas are highly vascular tumors, it is hypothesized that by applying the disrupting BBB agent to the tumor mass, efficient BBB disruption can be induced in the tumor mass as well as in the infiltrating zone. Thus, enabling efficient delivery of the systemically administered therapeutic agent by the tumors own vasculature to the target regions.

It is to be understood that the present invention is not limited to the treatment of brain tumors. The BBB disrupting agent disclosed in the invention may be further used for the treatment of a disease or disorder which would benefit from being combined with disruption of the BBB. These include neurodegenerative diseases, such as, Alzheimer's Disease, Multiple System Atrophy (MSA), Amyotrophic Lateral Sclerosis (ALS), and Parkinsonism (i.e., Parkinson's syndrome, atypical Parkinson's, or secondary Parkinson's, including Parkinson's Disease), among others. Each possibility represents a separate embodiment of the present invention.

According to some embodiments, the present invention provides a kit for increasing BBB permeability comprising at least one first container comprising a pharmaceutical composition comprising a modified serum albumin comprising serum albumin or an analogue thereof, said serum albumin comprises a plurality of neutralized amino acid side chain residues selected from Aspartic acid side chain residue, Glutamic acid side chain residue and a combination thereof, wherein each of said neutralized amino acid side chain residues is covalently attached to a capping moiety.

According to some embodiments, the present invention provides a kit for increasing BBB permeability comprising at least one first container comprising a pharmaceutical composition comprising a modified serum albumin comprising serum albumin, or an analogue thereof, said serum albumin comprises a plurality of cationized amino acid side chain residues selected from Aspartic acid side chain residue, Glutamic acid side chain residue and a combination thereof.

According to some embodiments, said serum albumin or an analogue thereof further comprises at least one therapeutic agent moiety covalently attached thereto through a lysine side chain residue.

According to some embodiments, the kit further comprises at least one second container comprising at least one therapeutic agent. According to some embodiments, the at least one therapeutic agent is an anti-neoplastic agent.

According to some embodiments, the kit further comprises instructions for use of said at least one first container. According to some embodiments, the kit further comprises instructions for use of said at least one second container. According to some embodiments, the pharmaceutical composition of said at least one first container is for intracranial administration. According to some embodiments, the pharmaceutical composition of said at least one first container is for intracranial administration by convection-enhanced delivery.

According to some embodiments, the kit further comprises an apparatus for convection-enhanced delivery. According to some embodiments, the kit further comprises instructions for performing convection-enhanced delivery. According to some embodiments, the kit further comprises instructions for coordinating the administration of each of said at least one first container and at least one second container. According to some embodiments, the kit further comprises a notice in the form described by a governmental agency regulating the manufacture, use or sale of pharmaceuticals or biological products, which notice reflects approval by the agency of manufacture, use or sale for human administration.

The term “an apparatus for convection-enhanced delivery administration” as used herein refers to any instrument required to practice the methods of the invention, such as a catheter, a syringe, a pump or any combination thereof.

The terms “comprises”, “comprising”, “includes”, “including”, “having” and their conjugates mean “including but not limited to”. The terms “comprises” and “comprising” are limited in some embodiments to “consists” and “consisting”, respectively. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure. In the description and claims of the application, each of the words “comprise” “include” and “have”, and forms thereof, are not necessarily limited to members in a list with which the words may be associated.

The examples hereinbelow are presented in order to more fully illustrate some embodiments of the invention. They should, in no way be construed, however, as limiting the broad scope of the invention. One skilled in the art may readily devise many variations and modifications of the principles disclosed herein without departing from the scope of the invention.

### Examples

### Example 1: The Potency of HSA Analogues to Disrupt an In-Vitro BBB Model

General Procedure for HSA Modified Analogues Preparation:

HSA (67 mg, 1 μmole) dissolved in 2 ml of H₂O containing 1M of glycine amide, alanine amide, leucine amide, ethylamine propylamine or ethanol amine. The pH was adjusted to pH 6.0±0.1. Excess of solid EDC (1-Ethyl-3-(3-dimethylaminopropyl) carbodiimide; 100 mg, 526 μmoles) was then added, and the reaction was carried out with stirring for 4 hr at 25° C. The obtained derivatives were dialyzed against H₂O for two days, with several replenishments of the H₂O, and then lyophilized. About 45 to 85 (out of 99) of the carboxylate moieties of all the analogues of HSA were modified by this procedure, resulting with transformation within the range of 40% to 90%. The modification was quantitated by reacting an aliquot of each analogue (˜2 mg) with 1M glycinamide, excess EDC, in 8M urea. Following dialysis, the additional glycine moieties were quantitated by amino acid analyses following acid hydrolyses. The protein concentration was calculated according to alanine (62 residues) and valine (41 residues).

Porcine Brain Endothelial Cells Monolayer Preparation and Transendothelial Electrical Resistance (TEER) Measurements:

Primary cultures of porcine brain endothelial cells monolayer (PBEC-M) were used as a cellular barrier. In brief, cells were isolated from freshly collected porcine brains as described previously (Cooper et al., J Neurochem 2011, 116: 467-475). Culture purity was confirmed by specific staining for Von-Willebrand factor. PBEC were seeded at a density of 100,000 PBEC/cm² on a microporous membrane of a Transwell insert placed into a 12 well plates. Cells were cultured in plating medium for up to 3 days until reaching confluence. Plating medium was composed of newborn calf serum (10%), L-glutamine (2 mM), penicillin (100 units/ml), streptomycin (0.1 mg/ml) and gentamicin (0.1 mg/ml), all dissolved in Earl's Medium 199. The medium was replaced with a serum-free medium (assay medium) for an additional period of 24-48 hr. The assay medium consisted of L-glutamine (2 mM), penicillin (100 units/ml), streptomycin (0.1 mg/ml), gentamicin (0.1 mg/ml) and hydrocortisone (550 nM) in Dulbecco-modified Earls medium (DMEM) diluted 1:1 in Hams F12 medium. The integrity of this cellular barrier was determined by measuring TEER, which reflects the impedance to the passage of small ions through the physiological barrier and is recognized as one of the most accurate and sensitive measures of BBB integrity. A decrease in TEER reflects increase impermeability and a loss of barrier function. TEER of the filter insert was recorded using an Endohm chamber connected to an EVOM resistance meter. The effective TEER of each filter insert was calculated by subtracting the TEER of the microporous membrane without PBEC and is reported in units of Ωcm². For testing the effects of the different modified HSA compounds on TEER, they were diluted in assay medium at the desired concentrations, and added to the luminal (to mimic blood to brain passage) or abluminal (to mimic brain to blood passage) side of the inserts.

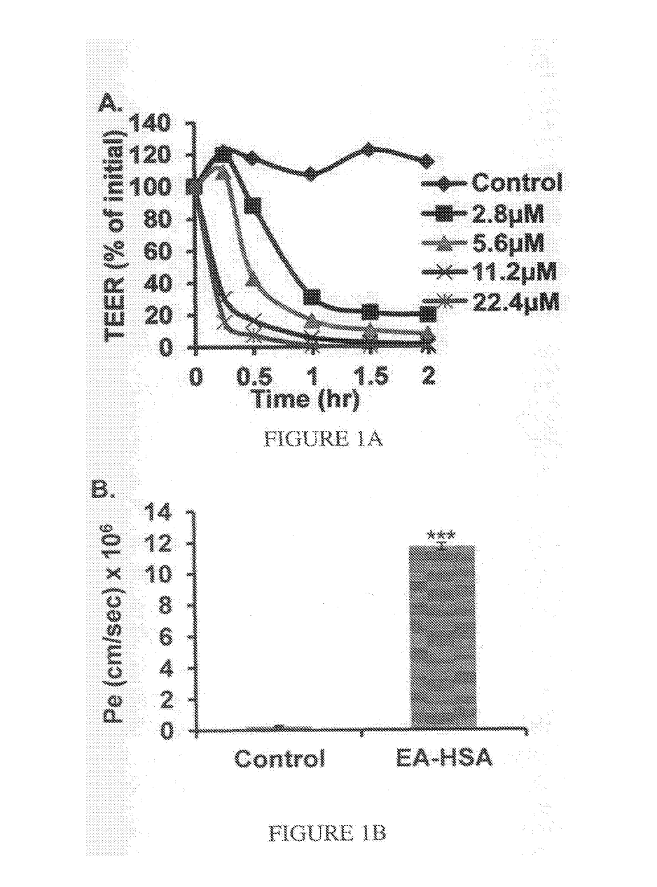

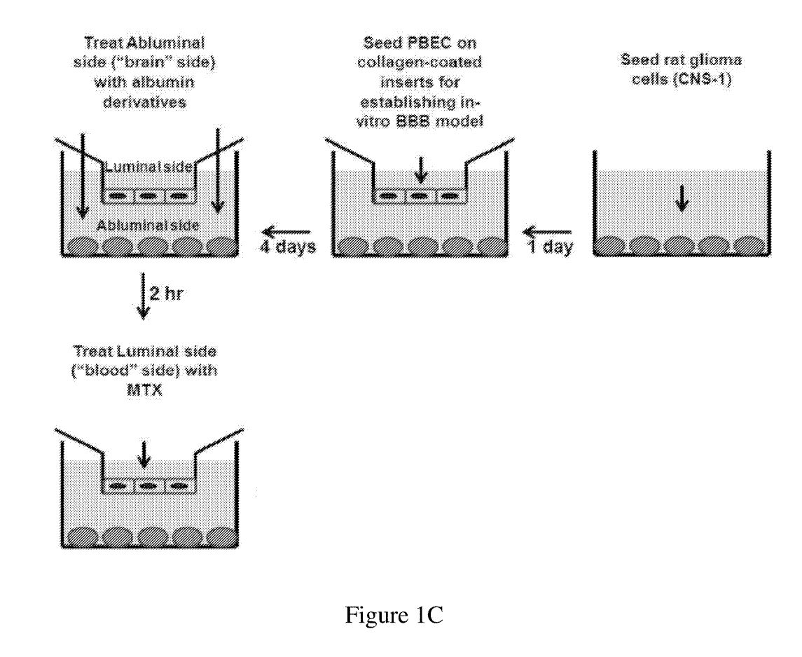

The following six modified HSA analogues were tested for their potency to disrupt the BBB in the in-vitro BBB model: HSA in which 85 carboxylic moieties were linked to glycine amide (Gly₈₅-HSA); HSA in which 83 carboxylic moieties were linked to leucine amide (Leu₈₃-HSA); HSA in which 80 carboxylic moieties were linked to ethylamine (EA-HSA); HSA in which 78 carboxylic moieties were linked to alanine amide (Ala₇₈-HSA); HSA in which 79 carboxylic moieties were linked to propylamine (PA₇₉-HSA); HSA in which 80 carboxylic moieties were linked to ethanol amine (E-Alco-HSA). As shown in Table 1, all the screened analogues disrupted the PBEC-M. EA-HSA was further evaluated and shown, at a concentration as low as 5.6 μM, to reduce TEER value by 90-98% within a short period (FIG. 1A).

TABLE 1
HSA analogues potency to disrupt an in-vitro BBB model

| | Modified HSA derivative | | | | | | | | | | | |
|---|---|---|---|---|---|---|---|---|---|---|---|---|
| | Gly₈₅-HSA | | Leu₈₃-HSA | | EA-HSA | | Ala₇₈-HSA | | PA₇₉-HSA | | E-Alco-HSA | |
| | (mg/ml) | | (mg/ml) | | (mg/ml) | | (mg/ml) | | (mg/ml) | | (mg/ml) | |
| | 1 | 0.1 | 1 | 0.1 | 1 | 0.1 | 1 | 0.1 | 1 | 0.1 | 1 | 0.1 |
| Max. BBB | 0 | 15 | 0 | 47 | 0 | ND | 1 | 78 | 2 | 97 | 4 | ND |
| opening after | | | | | | | | | | | | |
| 2 hrs (% of | | | | | | | | | | | | |
| initial TEER) | | | | | | | | | | | | |
| Time to reach | 30 | 120 | 30 | 120 | 45 | ND | ~60 | 120 | ~75 | 120 | ~90 | ND |
| max effect | | | | | | | | | | | | |
| (min) | | | | | | | | | | | | |
| T_{1/2} (min to | 5-15 | 60-120 | 5-15 | 60-120 | 5-15 | ND | 5-15 | — | 30 | — | 30 | ND |
| reach half | | | | | | | | | | | | |
| initial TEER) | | | | | | | | | | | | |

ND: not done; % of initial TEER - 0 indicates maximal opening of the BBB.

### Example 2: Permeability Studies In-Vitro Using Normalized Serum Albumin

To evaluate the ability of MTX to permeate BBB, the in-vitro BBB model described in Example 1 was employed. BBB inserts were treated for 2 hr at the abluminal side with EA derivatized HSA (14 μM; right column) or assay medium (left column) serving as control. MTX (1 mM) was placed at the luminal side, and the amount reached the abluminal side, in the presence and the absence of the EA-HSA, was quantitated by absorption at 305 nm, using ε₃₀₅=22,700. Permeability values were calculated as previously described (Cohen-Kashi Malina et al., Brain Res 2009, 1284: 12-21). Results are presented as mean±SEM (n=3-5 inserts per treatment).

As shown in FIG. 1B, modified albumin (EA-HSA) yielded a permeability value for the penetration of MTX of 11.74±1.3×10⁻⁶ cm/second (***p<0.001) which is a 47 fold increase relative to control which had permeability value of 0.25±0.05×10⁻⁶ cm/second.

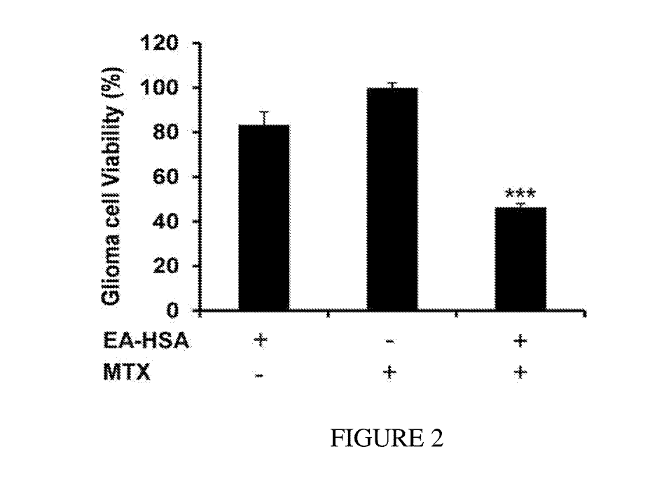

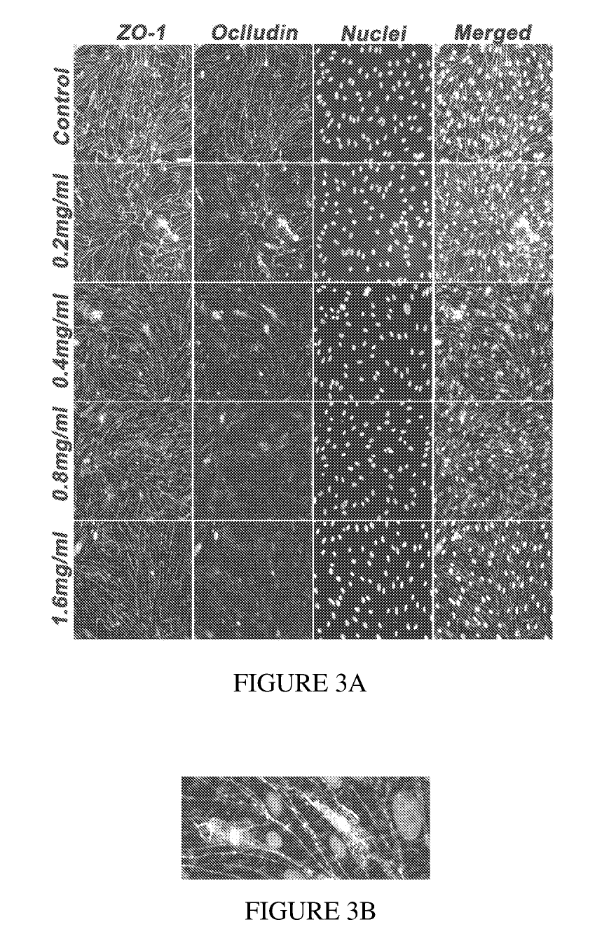

The antineoplastic efficacy of EA-HSA against glioma cells located in the brain side further was validated in the “brain cancer-related” in vitro BBB model (FIGS. 1C and 3). PBEC-M were treated with MTX in the presence and absence of EA-HSA. Results are presented as mean±SEM (n=4 inserts per treatment). FIGS. 1B and 2 establish that EA-HSA enables MTX entry at a sufficient rate (FIG. 1B) resulting with 50% cell death in the abluminal located glioma cell, within 48 hours (FIG. 2; ***p<0.001).

A schematic representation of the aforementioned assay for determining BBB permeability is shown in FIG. 1C.

### Example 3: The Effect of EA-HSA on Expression of TJ Related Membrane Proteins

The mode of action by which EA-HSA induces BBB permeability was investigated in a set of immunocytochemistry studies. These studies were performed to identify alterations in tight-junction (TJ)-related membrane protein(s). The in-vitro BBB assay described in example 1 was employed. The assays were carried out at a stage when TEER has been reduced by EA-HSA, to a level permitting the paracellular (between adjacent cells) passage of impermeable substances.

In the immunocytochemistry studies, PBEC were grown on Transwell inserts for several days until confluence was reached (TEER>300 Ωcm²). The cells were then fixed with ice cold 4% para-formaldehyde for 10 min at 25° C. and exposed to blocking solution (20% horse serum/0.1% Triton/phosphate-buffered saline (PBS)) for 2 hr. The PBEC were then incubated with mouse anti-occludin and rabbit anti ZO-1 antibodies at a 1:200 dilution, overnight at 4° C., washed with PBS and stained with a Cy3-labeled anti-rabbit or Alexa-Flour 488 anti-mouse secondary antibodies (1:200, 1 hr, RT). Nuclei were counterstained with Hoechst reagent for 20 sec. After mounting (Aqua Poly/Mount), the inserts were observed and photographed. Actin filaments were stained with Alexa Fluor 488-conjugated phalloidin (3 μl/insert, incubated together with the secondary antibody).

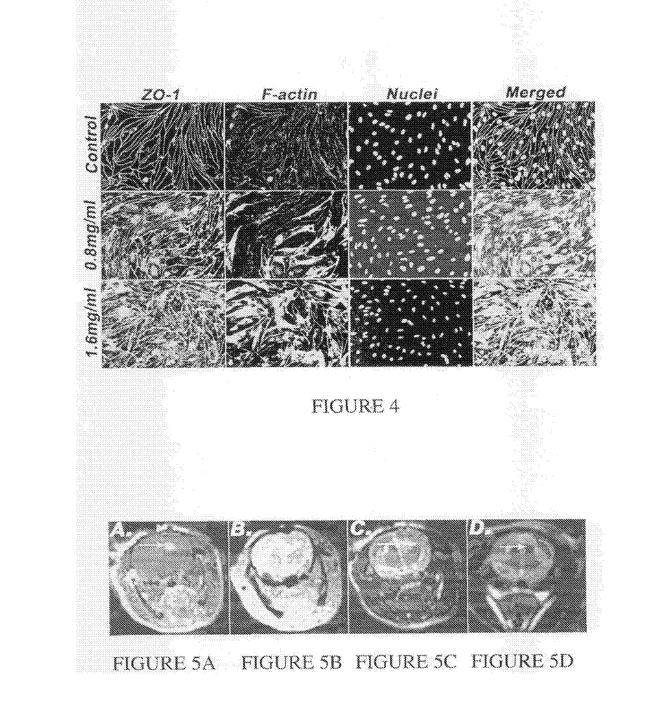

FIGS. 3 and 4 summarize the alterations in the PBEC-M following treatment with increasing concentrations of EA-HSA which permit paracellular entry of MTX.

The expression of Occludin, a major TJ protein responsible for the blockade of paracellular passage of molecules, was significantly altered following incubation with EA-HSA. Occludin HSA migrated from its location at the cell borders into the cytoplasm and degraded there (FIGS. 4A and 3B).

Zonula occludens-1 (ZO-1) is a scaffolding protein responsible for the linkage between the intracellular actin cytoskeleton and the outer membrane TJ's proteins (claudin-5 and occludin). This interaction is postulated to provide additional rigidity to the structures and allow for rapid alterations in barrier integrity in response to a variety of stimuli. As exemplified in FIG. 3A, the expression pattern of ZO-1 was only slightly altered upon incubation of PBEC-M with EA-HSA. The protein preserved its membrane location and showed minor alterations at high concentrations of EA-HSA.

Actin reorganization plays an important role in the cells structural support and may also play an active role in the formation and maintenance of TJ expression and patterns of distribution. FIG. 4 presents disorganization of the actin filaments, supporting the notion that the actin fibers have some role in the process of EA-HSA-induced BBB permeability.

Overall, the results demonstrate that out of the several transmembrane and cytosolic tight junction proteins being responsible to connect neighboring endothelial cells to each other, occludin expression was particularly altered upon incubation of PBEC-M with EA-HSA (FIG. 3). Without wishing to be limited by any theory or mechanism, the relatively unchanged ZO-1 may be explained by the cells attempt to maintain BBB functionality under the stress induced by the EA-HSA, also manifested by the formation of stress fibers (FIGS. 3 and 4). However, it should be noted that the exact mechanism leading to BBB opening may depend on the stress/compound imposed.

The aforementioned immunocytochemistry observations may explain the reduced PBEC-M tightness (FIG. 1) and the passage of impermeable agents such as MTX (FIG. 2). Without wishing to be bound by any theory or mechanism, the enhanced permeability under EA-HSA treatment was achieved by the occluding disruption with disorganization of cytoskeleton actin filaments.

### Example 4: Intracranial-CED Administration of EA-HSA In Vivo

The intracranial-CED administration of EA-HSA was detected in Lewis male rats using MRI. The experiments were conducted according to the recommendations of the declarations of Helsinki and Tokyo and to the Guidelines for the Use of Experimental Animals of the European Community approved by the Animal Care Committees of Sheba Medical Center. The experiments were performed with 29 rats weighing 250-300 g, 8-10 weeks old, fed on Purina Chow and water ad libitum. Ambient temperature was set to 22-23° C. with day/night light control.

The BBB disruption was assessed in normal rat brain by MRI. EA-HSA (20 μg/rat) was administered by CED into naïve (normal) rat brains under full anesthesia. The MRI contrast agent Gd-DOTA was administered intraperitoneally prior to CED (1 mmol/kg body weight). The first MRI series of images was acquired 30 min after EA-HSA was administered, another series of MRI scans was performed on day 7 to assess possible tissue damage. All rats were scanned under full anesthesia using a clinical GE 1.5 T MRI system with a clinical phased array knee coil and the following sequences: contrast-enhanced T1-weighted MRI for depiction of BBB disruption and assessing tumor volumes; T2-weighted MRI for assessment of early and late toxicity; and gradient echo (GE) MRI for depiction of possible hemorrhages.

FIGS. 5A-5D summarize a representative set of MRI scans acquired after CED administration of EA-HSA: T1-weighted MR images acquired 30 min after treatment (FIG. 5A); Gradient echo MR image acquired immediately post treatment (FIG. 5B); T2-weighted image acquired immediately following treatment (FIG. 5C); and T2-weighted image acquired 7 days following treatment (FIG. 5D). The scans reflect BBB-disruption (FIG. 5A, indicated by arrows), lack of hemorrhages (FIG. 5B) and tissue damage (FIG. 5C) and the lack of tissue toxicity following one week (FIG. 5D). T2-weighted MR images acquired immediately post CED (FIG. 5C) show enhancement in the treated region induced by the convective distribution of the infusate.

The results indicate that EA-HSA-induced BBB disruption is a transient phenomenon in vivo (FIG. 5). It seems that the BBB reverted back to its native-impermeable state within a short period following a single challenge with EA-HSA. Without wishing to be limited by any particular theory or mechanism of action, reformation of an impermeable status is likely due to denovo synthesis of occludin.

### Example 5: Combined EA-HSA and MTX Therapy In Vivo

The effect of combined intracranial-CED administration EA-HSA and systemically MTX therapy was examined in Lewis male rats bearing brain-glioma (CNS-1) tumor. The experiment design is summarized in Table 2.

Intracranial inoculation of the tumor was performed as follows: a midline scalp incision was carried out under general anesthesia in order to locate the bregma. A burr hole (1 mm) was drilled on the right side, 3 mm anterior and 2 mm lateral to the bregma. A 33-gauge needle attached to a 1,000 μl syringe was placed stereotactically into the striatum to a depth of 5 mm through which a pellet of 2×10⁵ CNS-1 rat glioma cells precipitated in 10 μl PBS buffer was infused into the striatum. The infusion was performed with a BASI syringe pump at a rate of 2 μl/min over a period of 5 min. The burr hole was sealed with bone wax to avoid the tumors from growing out of the skull. The intracranial inoculation of the tumor is designated Day-4 in Table 2.

EA-HSA was administered by CED into the rat brains under full anesthesia. For CED, a midline scalp incision was made under anesthesia to identify the bregma. Then, a burr hole (1 mm) was made in the right region of the skull, 3 mm anterior and 2 mm lateral to the bregma. For the tumor-bearing rats the previously made burr hole was re-opened. A 33-gauge needle attached to a 1,000 μL syringe was placed stereotactically 5.5 mm deep into the striatum. The infusion of EA-HSA was carried out with a BASI syringe pump at a rate of 2 μL/min for a period of 20 minutes.

Rats having glioma cells tumor were scanned 5 days post inoculation (Day 0 in Table 2), by contrast-enhanced MRI to determine tumor volumes and were divided into three groups of similar tumor volume distributions (n=12 per group). All groups were treated with a first composition intracranially (by CED) and then with a second composition intraperitoneally (IP), as follows: Group I: 10% sucrose in saline CED and saline IP; Group II: 10% sucrose in saline CED and MTX IP (6 mg/kg weight); and Group III: EA-HSA at 0.5 mg/ml by intracranial CED and 10% sucrose in saline CED and MTX IP (systemic administration). Follow-up MRI scans were accumulated on day 7 for assessing tumor volume (Day 2 in Table 2). Rats were also treated with MTX IP two and four days after the first MTX treatment.

It is to be emphasize that to increase the distribution efficacy of the infusates, the viscosity of the solutions delivered by CED was raised by adding 10% sucrose.

Additionally, MTX was administered intraperitoneally to naïve rats (n=26) at a dosage of 6 mg/kg body weight on days 0, 2 and 4. This mode of administration was found inappropriate as it resulted with symptomatic toxicity (reflected by weight loss, diarrhea, mucositis and shaggy fur) within a short period after administration. This symptomatic toxicity was fully avoided upon including folinic acid (leucovorin) into the treatment regimen (injected at 8 mg/kg weight). Naive rats (n=3) treated by the same MTX protocol with the addition of folinic acid (FA, Table 2) showed no toxicity—they gained weight according to normal weight standards and no visible chemotherapy-dependent symptoms were observed over a period of two weeks. The combined administration, MTX even at three times of its maximal tolerated dose, with folinic acid prevented MTX toxicity.

Without wishing to be limited by any particular theory or mechanism of action, it is hypnotized that folinic acid, a derivative of folic acid, enters the folate pathway downstream to dihydrofolate-reductase (DHFR). DHFR has a central role in DNA precursor synthesis, thus, it is considered as a target in cancer treatment by competitive inhibitor of DHFR, such as, MTX. Inhibition of this enzyme can limit the growth and proliferation of cells that are characteristic of cancer. Thus, despite the inhibition of DHFR by MTX, the synthesis of purines and pyrimidines and the maintenance of other folate dependent metabolic pathways are unaffected.

The volume (in mm³) of BBB disruption and the increase in tumor volume were calculated from T1-weighted MR images. Regions of interest (ROIs) were defined over the entire enhancing region for each slice (excluding the ventricles). The number of pixels in the ROIs was then counted and multiplied by the volume of a single pixel (voxel). Tumor growth rates were calculated by dividing the tumor volumes at days 2 and 7 post treatment with the baseline tumor volumes (measured at day 0).

TABLE 2
Efficacy experiment design: Groups, treatments and time line.

| Group | Day −4 | Day 0 | Day 1 | Day 2 | Day 3 | Day 4 | Day 5 |
|---|---|---|---|---|---|---|---|
| I-Control | Tumor | Saline CED/ | Saline | Saline | Saline | Saline | Saline |
| | inoculation | Saline IP | | | | | |
| II-MTX | Tumor | Saline CED/ | FA | MTX | FA | MTX | FA |
| | inoculation | MTX IP | | | | | |
| III-MTX + | Tumor | EA-HSA | FA | MTX | FA | MTX | FA |
| EA-HSA | inoculation | CED/ | | | | | |
| | | MTX IP | | | | | |

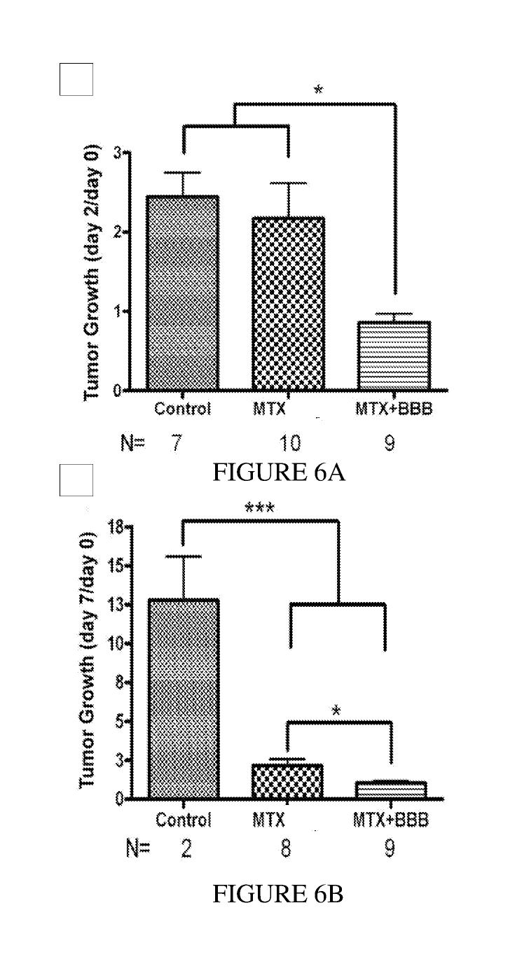

FIGS. 5 and 6 summarize the outcome of the combined treatment in the glioma rat model.

As demonstrated in FIGS. 6A-6B, untreated rats (control) showed increased average tumor volumes by a factor of about 2.4±0.3 within the first two days post treatment and an increase by a factor of about 12.8±2.8 within one week. Rats treated with MTX and EA-HSA (FIG. 6: MTX+BBB) fully suppressed tumor growth, leaving it nearly at the volume maintained at day 0 (0.9±0.1 and 1.0±0.1 for day 2 and day 7, respectively). Systemic (IP) administration of MTX did not result with a significant suppression in tumor size at 2 days post treatment (mean values 2.2±0.4 and 2.5±0.3 for the MTX-treated and control groups respectively, FIG. 6A). However, MTX alone exhibited a significant effect on tumor growth rate between day 2 and 7 relative to control (mean values 2.2±0.4 and 12.8±1.5 in the MTX and the control groups respectively, FIG. 6B). This observation suggests that the BBB turns leaky (and permeable to MTX) when tumor volume increased radically (×2.5) reaching above 86.1±13.6 mm³, at day 2.

The combined treatment of MTX via systemic administration together with EA-HSA, via intracranial CED, fully suppressed tumor growth, leaving it nearly at the volume measured at day 0 (0.9±0.1 and 1.0±0.1 for day 2 and day 7 respectively).

The results of the efficacy study in the rat glioma model showed that despite the rapidly growing tumors in the control group, the combined therapy completely suppressed tumor growth. Interestingly, systemic administration of MTX seemed to show no therapeutic effect in the first 2 days post treatment (no significant change in tumor growth versus control) while the combined approach showed significant anti-tumor effects (complete arrest in tumor growth) at that time point. Later on, 7 days post treatment, systemically administered MTX did show therapeutic benefits, although still significantly lower than those obtained by the combined therapy. This delayed effect of MTX may be explained by increased BBB disruption as the tumor matures or by an accumulated effect on the tumor vasculature induced by repeated treatments with MTX. It also suggests that in the group receiving the combination therapy, where the tumor failed to grow, MTX entry was highly dependent on the BBB-opening efficacy of EA-HSA.

The three rats groups were monitored for survival. Rats were monitored daily and sacrificed when lost>20% of body weight and/or were unable to eat or drink. Rats were monitored up to 60 days after the tumor cell implantation. The Kaplan-Meier survival curves were analyzed according to the Wilcoxon test.

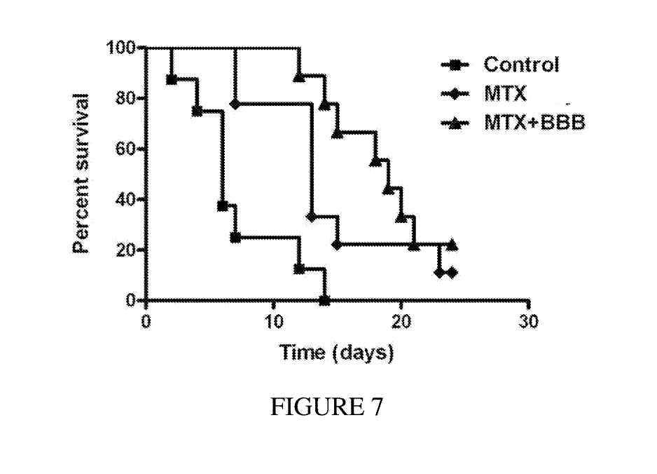

A Kaplan-Meier analysis indicates that the median survival times of rats bearing intracranial CNS-1 glioma tumors treated with combined treatment (FIG. 7; MTX-BBB; triangle; P<0.001) amounted to 19 days compared to the 12 days of MTX alone treated rats (FIG. 7; MTX; diamond; P<0.001). The median survival time of untreated rats was 5 days (FIG. 7; Control; square). The combined therapy significantly prolonged survival by nearly a factor of 3 compared to control.

### Example 6: BBB Disruption In Vitro by Cationized-HSA Analogues

The effect of cationized-HSA analogues on the permeability of BBB was determined using an in-vitro BBB model as described in Example 1.

The general procedure for cationized-HSA analogues preparation was implemented according to the synthetic procedure in Example 1. The compounds used for cationizing Aspartic acid and Glutamic acid side chain residues were: 1,3 diaminopropane-2HCl, hexamethyldiamine-2HCl, Dicystamine-2HCl, argininamide-2HCl and ethylamine-HCl.

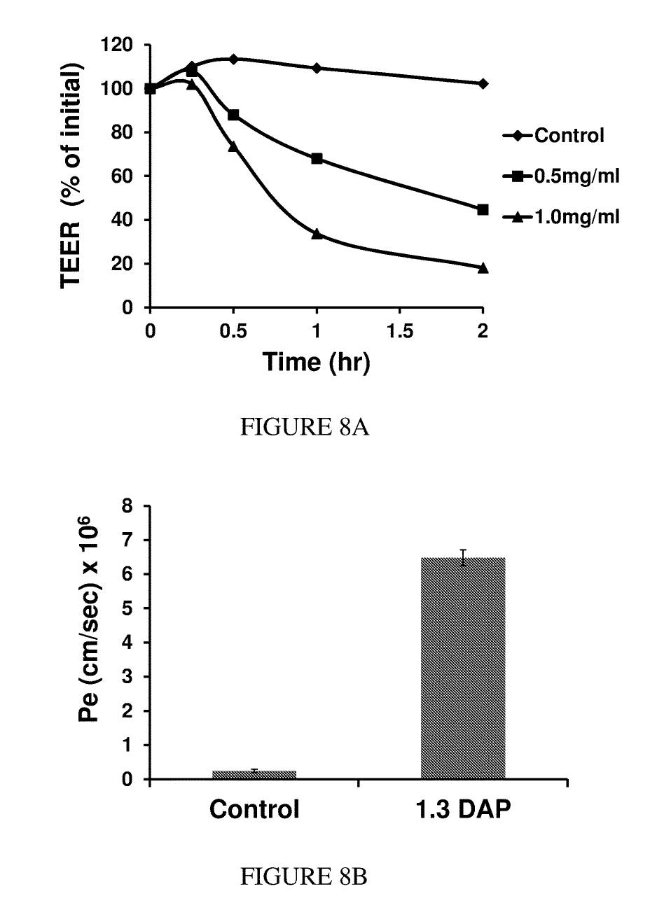

The following five modified HSA analogues were tested for their potency to disrupt the BBB in the in-vitro BBB system. Most of the screened molecules disrupted the PBEC-M, allowing penetration of impermeable agents at a concentration range of 3 to 30 μM. 1,3-DAP cationized HSA was further evaluated and showed, at a concentration as low as 1 mg/ml, to reduce TEER value by about 80% within a short period (FIG. 8A).

### Example 7: Permeability Studies In-Vitro Using Cationized Serum Albumin

To ability of MTX to permeate BBB was evaluated using the in-vitro BBB assay

(Example 1). BBB inserts were treated for 2 hr at the abluminal side with 1,3-DAP-cationized-HSA (14 μM; right column) or assay medium (left column) serving as control. MTX (1 mM) was placed at the luminal side, and the amount reached the abluminal side, in the presence and the absence of the cationized-HSA was quantitated by absorption at 305 nm, using ε₃₀₅=22,700. Permeability values were calculated as previously described (Cohen-Kashi Malina et al., ibid). Results are presented as mean±SEM values (n=3 inserts per treatment).

As shown in FIG. 8B, the 1,3-DAP-cationized-HSA yielded a permeability value of 6.48±0.23×10⁻⁶ cm/second (***p<0.001) for the penetration of MTX.

### Example 8: Intracranial-CED Administration of 1,3-DAP-Cationized-HSA In Vivo

The effect of intracranial-CED administration of 1,3-DAP-cationized-HSA was evaluated in Lewis male rats using MRI technology as described in Example 4.

Briefly, 1,3-DAP-cationized-HSA (40 μg/rat) was infused by CED into naïve rat brains under full anesthesia. The MRI contrast agent Gd-DOTA was administered intraperitoneally prior to CED (1 mmol/kg body weight).

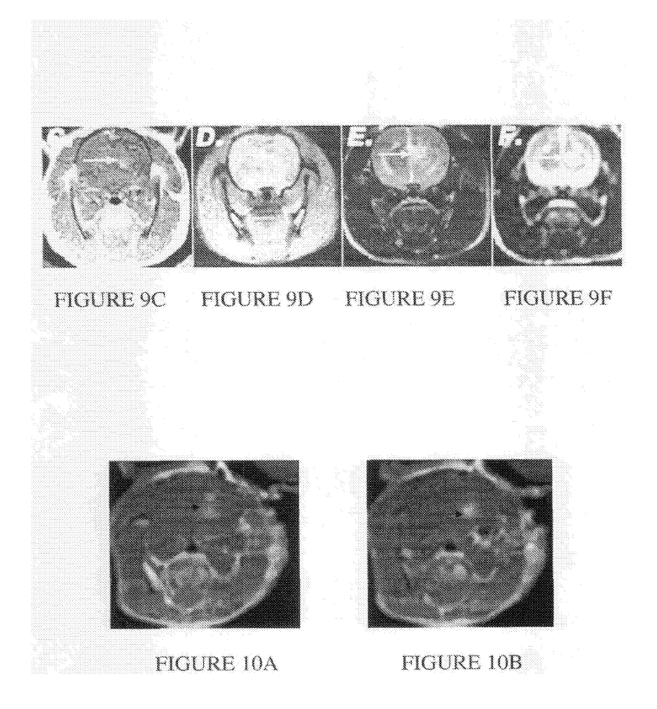

FIG. 9 summarizes a representative set of MRI scans acquired after CED administration of 1,3-DAP-cationized-HSA. Shown are T1-weighted MR images acquired 30 min after treatment (FIG. 9C); Gradient echo MR image acquired immediately post treatment (FIG. 9E); T2-weighted image acquired immediately following treatment (FIG. 9D); and T2-weighted image acquired 7 days following treatment (FIG. 9F).

The scans reflect BBB-disruption (FIG. 9C, indicated by arrows), lack of hemorrhages (FIG. 9D) lack of tissue damage (FIG. 9E) and the lack of tissue toxicity following one week (FIG. 9F). The T2-weighted MR images acquired immediately post CED (FIG. 9E) show enhancement in the treated region induced by the convective distribution of the infusate.

### Example 9: Intracranial-CED Administration of HSA-Gly-MTX In Vivo

HSA in which 85 carboxylic moieties were linked to glycine amide and 3 lysine side chains were linked to MTX (HSA-Gly₈₅-MTX₃) was prepared according to the steps detailed below.

### Preparation of MTX-Anhydride

Methotrexate (MTX; 45.4 mg, 100 μmoles) was dissolved in 0.9 ml dimethyl sulfoxide (DMSO) and 95 μl of a 1M DCC (dicyclohexyl carbodiimide) solution of in DMF (dimethyl formamide; 95 μmoles) was then added. The reaction was carried out for 2 hrs at 25° C. Dicyclohexylurea was removed by filtration. The MTX-anhydride formed was kept at 4° C.

### Preparation of HSA-Gly₈₅-MTX₃

HSA-Gly₈₅ (also termed Gly₈₅-HSA hereinabove), 71 mg (1 μmole) was dissolved in 2 ml of 0.05M Hepes buffer (pH 7.4) and cooled to 0° C. Ten aliquots of MTX-anhydride, 10 μl each, from a solution of 100 μmole/ml in DMSO were then added to the stirred solution over a period of 1 hr (10-fold excess over the protein derivative). The reaction proceeded an additional hour and then dialyzed over a period of 3 days at 4° C., first against 0.1M NaHCO₃ (pH 8.5) for 1 day and then additional 2 days against H₂O. Subsequently the product was lyophilized. Using this procedure, about 3 to 4 mole-equivalents of MTX are incorporated to the lysine moieties of this protein derivative, as determined by its absorbance at 372 nm using ε₃₇₂=7200. The protein concentration was determined by acid-following by quantitative amino acid analysis according to alanine (62 residues) and valine (41 residues). This procedure is also suitable for preparation of HSA-Gly₈₅-MTX₃.

Intracranial-CED administration of HSA-Gly₈₅-MTX₃ in vivo:

The intracranial-CED administration of HSA-Gly₈₅-MTX₃ was performed in Lewis male rats and detected using MRI. The experiments were conducted according to the recommendations of the declarations of Helsinki and Tokyo and to the Guidelines for the Use of Experimental Animals of the European Community approved by the Animal Care Committees of Sheba Medical Center. The experiments were performed according to the procedure of Example 4.

In brief, an infusion of HSA-Gly₈₅-MTX₃ (40 μg/rat) was administered by CED into naïve (normal) rat brains under full anesthesia. The MRI contrast agent Gd-DOTA was administered intraperitoneally prior to CED (1 mmol/kg body weight). The first MRI series of images was acquired 30 min after the conjugate was administered. Another series of MRI scans was obtained on day 7 to assess possible tissue damage. All rats were scanned under full anesthesia using a clinical GE 1.5 T MRI system with a clinical phased array knee coil and the following sequences: contrast-enhanced T1-weighted MRI for depiction of BBB disruption and assessing tumor volumes; T2-weighted MRI for assessment of early and late toxicity; and gradient echo (GE) MRI for depiction of possible hemorrhages.

FIGS. 10A-10B are two representative MRI scans of different slices of a rat's brain acquired 30 minutes after CED administration of HSA-Gly₈₅-MTX₃. In the images the normal brain tissue, where the BBB is intact, appears gray. The contrast agent, which appears bright (white) in this type of images, is invisible if the BBB is intact since then the contrast agent is confined within the blood vessels. As such, the contrast agent does not penetrate the tissue due to the BBB which prevents it from leaking through the vessel walls into the tissue. However, where the BBB is disrupted by the HSA-Gly₈₅-MTX₃ conjugate, the contrast agent leaks through the vessels walls and accumulates in the extracellular region of the surrounding tissue, thus inducing enhancement (white region) in the MR images.

In order to detect the presence of fresh hemorrhages gradient-echo MRI was used, as it depicts susceptibility artifacts (dark regions in the images) induced by accumulation of blood.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-28. (canceled)

29. A modified serum albumin, comprising:
serum albumin, or an analogue thereof, having a plurality of neutralized amino acid side chain residues selected from the group consisting of Aspartic acid side chain residue, Glutamic acid side chain residue, and a combination thereof;
wherein each of said plurality of neutralized amino acid side chain residues is covalently attached to a capping moiety.

30. The modified serum albumin of claim 29, wherein the serum albumin includes human serum albumin.

31. The modified serum albumin of claim 29, wherein the capping moiety includes a nitrogen containing substituent.

32. The modified serum albumin of claim 31, wherein the capping moiety is selected from the group consisting of glycine amide, alanine amide, leucine amide, ethylamine, propylamine, and ethanol amine.

33. The modified serum albumin of claim 32, wherein the capping moiety is ethylamine.

34. The modified serum albumin of claim 29, wherein the plurality of neutralized amino acid side chain residues include at least 60 neutralized amino acid side chain residues.

35. The modified serum albumin of claim 29, further comprising at least one therapeutic agent moiety covalently attached to the albumin through a lysine side chain residue, thereby producing a conjugate.

36. The modified serum albumin of claim 35, wherein the at least one therapeutic agent moiety includes an anti-neoplastic agent.

37. A pharmaceutical composition, comprising:
a cationized serum albumin or an analogue thereof, said cationized serum albumin includes a plurality of cationized amino acid side chain residues selected from the group consisting of Aspartic acid side chain residue, Glutamic acid side chain residue, and a combination thereof; and
pharmaceutically acceptable diluents or carriers.

38. The pharmaceutical composition of claim 37, wherein the cationized serum albumin includes at least one therapeutic agent moiety covalently attached to the albumin through a lysine side chain residue, thereby producing a conjugate.

39. The pharmaceutical composition of claim 38, wherein the at least one therapeutic agent moiety includes an anti-neoplastic agent.

40. A method for increasing blood-brain barrier permeability in a subject in need thereof, the method comprising administering to the subject a pharmaceutical composition comprising the modified serum albumin of claim 29.

41. The method of claim 40, wherein administering includes administering the pharmaceutical composition intracranially by convection-enhanced delivery.

42. The method of claim 40, further comprising administering to said subject at least one therapeutic agent.

43. The method of claim 42, wherein the at least one therapeutic agent moiety includes an anti-neoplastic agent.

44. A method for treating a disease or disorder in a subject in need thereof, the method comprising administering to the subject the modified serum albumin of claim 29.

45. The method of claim 44, wherein administering includes administering the modified serum albumin intracranially by convection-enhanced delivery.

46. A method for increasing blood-brain barrier permeability in a subject in need thereof comprising administering to the subject the pharmaceutical composition of claim 37.

47. The method of claim 46, further comprising administering to said subject at least one therapeutic agent.

48. The method of claim 47, wherein the at least one therapeutic agent is selected from the group consisting of anti-neoplastic agents, anti-angiogenic agents, siRNAs, immuno-therapeutic agents, and chemotherapeutic agents.
